(19) United States
(12) Patent Application Publication
(10) Pub. No.: US 2016/0041086 A1
(43) Pub. Date: Feb. 11, 2016

(54) LUBRICATION CONTROL SYSTEM AND RELATED METHOD OF USE

(71) Applicant: Innovative Cleaning Equipment, Inc., Grand Rapids, MI (US)

(72) Inventors: Nicholas D. Jacques, East Grand Rapids, MI (US); William N. Sneller, Grand Rapids, MI (US); Luke T. Ganzevoort, Caledonia, MI (US)

(21) Appl. No.: 14/454,058

(22) Filed: Aug. 7, 2014

(51) Int. Cl. G01N 19/02; F16N 29/02; B65G 45/02; G01N 33/28

(52) U.S. Cl. CPC G01N 19/02; G01N 33/2888; F16N 29/02; B65G 45/02

(57) ABSTRACT

A system for measuring and optionally controlling the COF of moving surfaces. The system may include a component that rides on the moving surface and a controller that measures the drag force between the component and the moving surface. The system may include a linkage to hold the component so that it does not move with the moving surface and a load cell to measure the force between the component and the moving surface. The load cell may be oriented transversely to the drag force. The system may control a lubrication system based on the measurements. The system may include a component that is urged into the moving surface by a normal force, and may include one load cell to measure the tangential force and another to measure the normal force. A controller may compare the measured tangential and normal forces to obtain a representation of the COF.

### BACKGROUND OF THE INVENTION

The present invention relates to systems for measuring the coefficient of friction of moving surfaces and more particularly to systems and methods for monitoring the coefficient of friction and potentially controlling application of lubrication.

In many systems where one object is in contract with a moving surface such as a conveyor belt it is necessary to apply a lubricant to a conveyor belt to improve the performance of the systems. One system in particular is an accumulator area in a bottling filling operation. In an accumulator, bottles are fed into a staging area where they are collected to form large quantities for packaging operations. Bottles move into the area at relatively high speeds, but are constrained from moving out of the area by a barrier. The bottles that have stopped must be adequately lubricated to prevent toppling, bottles that are still in motion must not be excessively lubricated or they will not reach the end of the table and join the accumulating group. Most systems have a range of acceptable lubrication levels. Various methods have been used in the past to apply lubricant to such a system within the allowable levels. One method is to have a person apply lubricant by visual inspection or at some semi-regular timer interval, such as the beginning of a shift. Another method is to apply a fixed amount of lubricant at regular intervals of time using a computer controlled timer. This method ensures a more regular application of lubricant, but is an open system with no feedback. Over time excess lubricant can accumulate or a lubricant deficiency can occur. These methods may allow lubricant to be over applied resulting in waste of lubricant and undesirable effects in the operation of the system due to excess lubricant, they also allow too little lubricant to be applied resulting in undesirable effect due to inadequate lubrication.

In an effort to maintain appropriate lubrication on conveyor it is not uncommon to manually obtain measurements representative of the coefficient of friction between a conveyor belt and products supported on the belt. Typically, the coefficient of friction is determined manually using measurements obtained with a fish scale or similar type of hand scale. In practice, the product is first measured using a fish scale to obtain its weight. This gives a number representative of the force normal to the conveyor belt. Next, a rope is secured to one or more products and to the fish scale, and the product is allowed to drag over the belt so that the drag force is measured by the scale. The weight measurements and drag force measurements can then be used to determine the coefficient of friction. This method is cumbersome, labor intensive and time consuming. As a result, it is impractical and not particularly well-suited for frequent use in a commercial setting.

### SUMMARY OF THE INVENTION

The present invention provides a system and method for measuring the coefficient of friction of moving surfaces, such as conveyor belts. In one embodiment, the COF monitoring system includes a monitor that measures the coefficient of friction between itself and a moving surface, and uses that measured value as a proxy for the lubrication level. The COF monitoring system may include a sled housing configured to ride on the moving surface, a linkage to hold the sled housing so that it does not move with the moving surface and a load cell operatively coupled between the sled housing and the linkage to measure the force between the moving surface and the stationary sled housing. In use, the drag force between the sled housing and the moving surface is measured by the load cell. The load cell measurements are representative of the coefficient of friction between the sled housing and the moving surface.

In one embodiment, the load cell is a strain gauge load cell that converts a force into an electrical signal representative of the force acting on the strain gauge. For example, the load cell may include a plurality of strain gauges arranged in a Wheatstone bridge (e.g. quarter-bridge, half-bridge or full-bridge). In one embodiment, the load cell includes one end that is fixed relative to the sled housing and another end that is coupled to the linkage. The linkage may include a flexible cable having one end that is attached to a structure that is fixed adjacent the moving surface, such as the framework of a conveyor, and another end joined to the free end of the load cell. The load cell may be oriented transversely within the sled housing. The linkage may be joined to the load cell by a centering bracket. The centering bracket brings the attachment point for the linkage to the approximate center of the housing, thereby helping to minimize canting of the sled housing on the moving surface.

In one embodiment, the system includes a controller that receives as an input the electrical signal generated by the load cell. The controller is configured to convert the electrical signals received from the load cell into a representation of the coefficient of friction encountered between the sled housing and the moving surface.

In one embodiment, the COF monitoring system includes a lubrication system used to apply lubrication to the moving surface, when appropriate as determined by the monitoring system. The lubrication may be integrated with the COF monitoring system or it may be a separate system that operates, at least in part, based on input from the COF monitoring system.

In one embodiment, the COF monitoring system determines the coefficient of friction or a number representative of the coefficient of friction. In one embodiment, the lubrications system applies lubricant only when the coefficient of friction exceeds certain preset values corresponding to the ideal range of lubrication levels. In the example of the accumulator table, a technician can observe when bottles begin to behave as if there is not enough lubricant in the system. The technician can then measure the coefficient of friction between the device and the conveyor belt using the invention, and choose a value slightly greater than the measured value as the minimum lubrication level. Second the technician can apply lubricant until the system behaves as if too much lubricant is applied. Once these values are known, the technician can set parameters within the device to automatically ensure that the minimum and excess lubrication values are not exceeded. This device allows even tighter control of lubrication within the acceptable range. One reason for such control is to reduce lubrication consumption which has benefits of cost savings, and reducing environmental impact.

In one embodiment, the present invention also provides a method of maintaining appropriate lubrication on a moving surface. In one embodiment, the method includes the steps of placing a sled housing on a moving surface, providing a fixed linkage for the sled housing, coupling the linkage to a load cell within the housing. The device functions by determining the drag force corresponding to the coefficient of friction exceeds the preset level. Once the determination has been made, the device applies a preset amount of lubricant. The device then allows the lubricant to disperse through the system. After a set time, the device begins to monitor the drag force again, and make a determination if more lubricant needs to be applied.

In an alternative embodiment, the COF monitoring system includes a monitor that measures the coefficient of friction between a weighted sled and the moving surface. In this embodiment, the COF monitoring system may include a housing configured to mount adjacent to a moving surface and a weighted sled that is coupled to the housing by a linkage. The linkage joins the sled to a load cell within the housing so that the load cell can provide measurements representative of the friction between the sled and the moving surface. In use, the load cell measures the drag force between the sled and the moving surface, which is representative of the coefficient of friction between the sled and the moving surface.

In an alternative embodiment, the COF monitoring system has a sled housing that is configured to engage an undersurface. This may be particularly useful in applications where objects on the upper surface are undesirable. For example, in this embodiment, the sled housing may be position underneath a conveyor system on a support structure that urges the sled housing upward into the returning conveyor belt. In this embodiment, the monitor includes one load cell that measures the tangential force (generally as described above), and one load cell that measures the normal force (e.g. the force at which the sled housing is urge into the moving surface). The system may include a controller that compares the tangential force to the normal force to obtain a representation of the COF between the sled housing and the moving surface. The controller may make a logical decision to adjust one or more parameters (e.g. apply lubrication) within the system that have an effect on the relationship between tangential force and normal force of the object and the moving surface within the system.

The present invention provides a simple and effective monitoring system that can be used to measure the coefficient of friction of a wide range of moving surfaces. The present invention includes alternative embodiments in which a load cell is capable of taking a measurement representative of the COF based on the drag force between the moving surface and an object riding on the moving surface. The present invention includes alternative embodiments in which the electronics are housed within a sled housing riding on the moving surface or in a housing mounted adjacent the moving surface. The sled housing requires a relatively small amount of space and can be configured to engage the surface from essentially any direction (e.g. top, bottom). The system can be easily integrated with other systems, such as lubrication systems capable of applying lubricant based on the coefficient of friction measurements. The use of an integrated load cell provides simple and consistent measurements. When included, the center bracket helps the sled housing to ride straight over the belt. In one embodiment, the electronics can be housed in a stationary housing and a sled may be placed on the moving surface. This can facilitate installation, use and maintenance. When desired, the sled housing can be urged into the moving surface to facilitate engagement. For example, a lift arm can be used to urge the sled housing into the moving surface when the force of gravity is not alone sufficient to do so. In such applications, the electronics may include two load cells that determine COF based on the drag force and the force urging the sled housing into the moving surface.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

FIG. 1 is a perspective view of a conveyor assembly incorporating a coefficient monitoring system in accordance with an embodiment of the present invention.

FIG. 2 is an exploded perspective view of the monitor.

FIG. 3 is a flow chart showing the general steps of a method for operating the present invention.

FIG. 4 is a perspective view of a conveyor system incorporating an alternative embodiment of the present invention.

FIG. 5 is an enlarged side view of the conveyor system of FIG. 4.

FIG. 6 is a perspective view of the coefficient monitoring system with portions of the housing removed.

FIG. 7 is a perspective view of a conveyor assembly incorporating a coefficient monitoring system in accordance with a second alternative embodiment of the present invention.

FIG. 8 is a perspective view of the coefficient monitoring system of FIG. 7.

FIG. 9 is a top perspective view of the monitor of FIG. 7.

FIG. 10 is a top perspective view of the monitor with the drag plate removed.

FIG. 11 is a top perspective view of the monitor with portions of the housing removed.

FIG. 12 is a top perspective view of the monitor with portions removed.

FIG. 13 is a bottom perspective view of the load cell and bracket assembly.

### DESCRIPTION OF THE CURRENT EMBODIMENT

Overview.

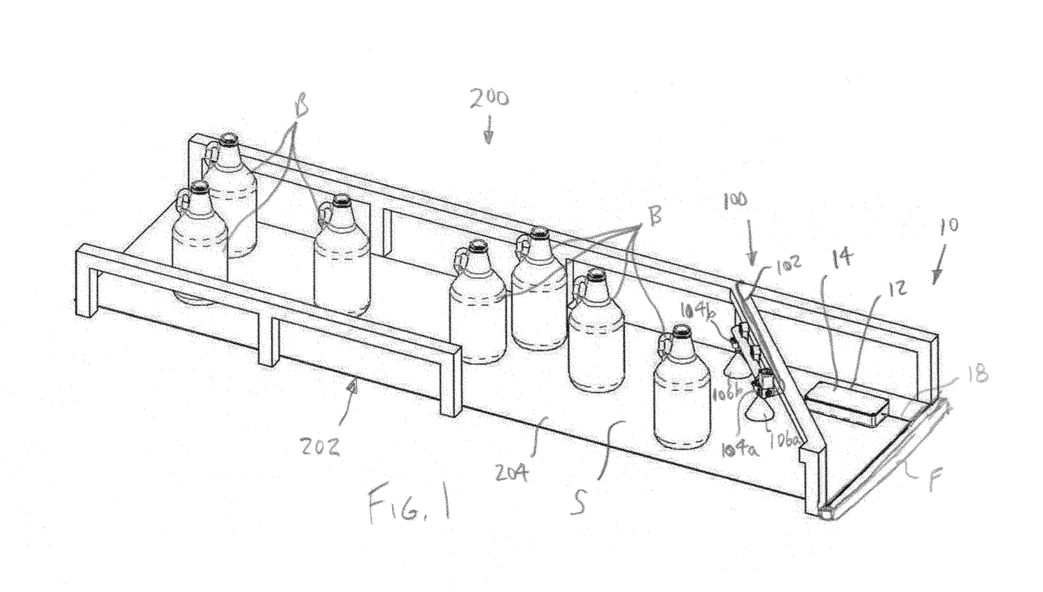

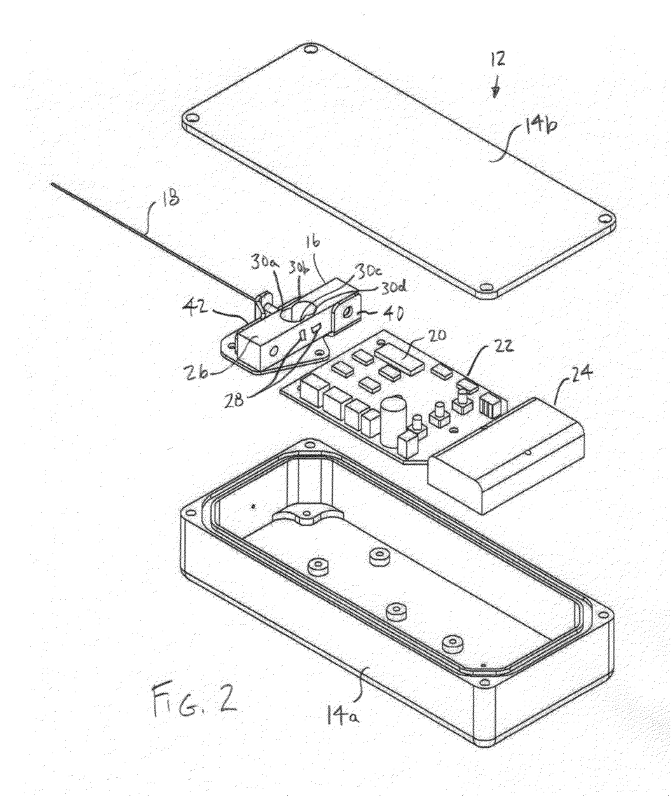

A coefficient of friction (COF) monitoring system in accordance with an embodiment of the present invention is shown in FIGS. 1 and 2. In this embodiment, the COF monitoring system 10 includes a monitor 12 having a sled housing 14 that rides on a moving surface S and is configured to obtain measurements representative of the coefficient of friction between the sled housing 14 and the moving surface S upon which it is riding. The monitor 12 of this embodiment includes a load cell 16 disposed within the sled housing 14. The load cell 16 is coupled to a fixed structure F by a linkage 18 so that the sled housing 14 is dragged over the moving surface S. More specifically, the linkage 18 holds the sled housing 14 as the moving surface S travels below. As such, the load cell 16 produces electrical signals representative of the drag force (and consequently the COF) between the sled housing 14 and the moving surface S.

The information obtained by the COF monitoring system 10 may be used for a variety of purposes. For example, the information may be collected and stored to provide a database of historical data. This database may be used to better understand operation of the system being monitored, such as how COF may affect wear and other operational parameters. As another example, the COF monitoring system 10 may be used in connection with realtime operation and control, such as realtime control over the application of lubricant to the moving surface or in triggering preventative maintenance or repair.

In the illustrated embodiment, the COF monitoring system 10 is operatively coupled to a lubrication system 100 that selectively applies lubrication to the moving surface S when appropriate as determined based on the COF measurements generated by the load cell 16. In one embodiment, the monitor 12 includes a controller 20 configured to generate an output signal capable of activating the lubrication system 100. In use, the COF monitoring system 10 may periodically determine the COF (or a number representative of COF) between the sled housing 14 and the moving surface S, and activate the lubrication system 100 when the determine COF passes a threshold value. The controller 20 may then enter a “delay” mode during which time the lubrication is allowed to become more evenly distributed over the belt. The length of time that the system 10 remains in delay mode may vary. Once the system 10 wakes from delay mode, it again begins monitoring COF to determine when to again apply additional lubrication.

In the illustrated embodiment, the amount of lubrication to be applied is predetermined and, when monitoring shows that lubrication is desired, the controller 20 is configured to operate the lubrication system 100 for that predetermined period of time. For example, the controller 20 may have an output that directly controls the lubrication system 100. Alternatively, the system 10 may use its COF monitoring capabilities to determine when sufficient lubrication has been applied. For example, the system 10 may activate the lubrication system 100 for a short period of time to dispense a small amount of lubrication on the moving surface, enter a delay period of time to allow the lubrication to spread and following the delay period measure the COF to determine if additional lubrication is desired. The process of applying, delaying and measuring can be repeated until the appropriate COF has been reached.

The present invention is described in the context of a conveyor system and, more particularly, the accumulator area of a belt-based conveyor system. The present invention is, however, well-suited and readily adapted for use in essentially any other application where it is desirable to measure the COF of a moving surface and optionally to automate a response to the COF measurements. Further, the present invention is described in connection with a lubrication system. The present invention may be configured to provide other automated responses to the COF measurements, such as logging measurements or interacting with alternative systems that are capable of adjusting COF or affecting controlled on the system based on COF.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

General Construction.

As noted above, the COF monitoring system 10 of the illustrated embodiment is installed on a conveyor system 200 and is used to monitor and control the application of lubricant to the top surface of the conveyor belt 204. In this application, the primary function of the system 10 is to maintain an appropriate coefficient of friction between the conveyor belt 204 and the objects carried on the conveyor belt 204, which in this embodiment are bottles B. Generally speaking, it is desirable for the COF of friction to be sufficiently low to allow the belt to move under an accumulated volume of bottles without tipping or upsetting the bottles, while at the same time being sufficiently high to allow the conveyor belt 204 to move the bottle B when appropriate. In this embodiment, the COF monitoring system 10 is installed in the accumulator section 202 of the conveyor system 200 to monitor the COF of the conveyor belt 204 and to selectively control the lubrication system 100 to maintain the appropriate COF between the belt 204 and the products carried on the belt.

The COF monitoring system 10 of the illustrated embodiment generally includes a monitor 12 having a sled housing 14 that contains an electronics module 22 and a linkage 18 that anchors the sled housing 14 to a fixed structure F. As such, the sled housing 14 rides on the moving belt held from moving with the belt 204 by the linkage 18. In the illustrated embodiment, the sled housing 14 is a generally rectangular box of sufficient size and shape to accommodate the electronics module 22. The housing 14 of the illustrated embodiment includes a base 14a and a cover 14b. The size, shape and configuration of the sled housing 14 may vary from application to application. The undersurface of the sled housing 14 in the illustrated embodiment is a simple plane with rounded edges that ease interaction between the sled housing 14 and the conveyor belt 204. The undersurface of the sled housing 14 may be manufactured from the same material as the objects that will be moved on the conveyor belt 204. This may provide improved correlation between the COF measurements taken by monitor 12 and the actual COF between the conveyor belt 204 and the objects carried by the belt 204 (e.g. bottles in FIG. 1). The undersurface of the sled housing 14 may be interchangeable and it may be selected from application to application to match with the objects carried by the belt 204.

The electronics module 22 generally includes a load cell 16, an amplifier 34, a controller 20 and a power source 24. The load cell 16 is operatively coupled to the linkage 18 so that the forces acting on the linkage 18 are communicated to the load cell 16. As a result, the load cell 16 produces electrical signals in response to the drag force produced by the moving surface S moving beneath the sled housing 14. The drag force is representative of the coefficient of friction between the sled housing 14 and the conveyor belt 204. In the illustrated embodiment, the load cell 16 is a strain gauge load cell that converts a force into an electrical signal that is representative of the force acting on the strain gauge. In the illustrated embodiment, the load cell 16 includes four strain gauges 28 mounted to a carrier 26 (although only two are visible in FIG. 2). The carrier 26 may be configured to include four thinned regions 30a-d that focus the forces acting on the carrier 26. As shown, the strain gauges 28 may be mounted to the carrier 26 adjacent to the thinned regions 30a-d. Although the illustrated embodiment includes four strain gauges, the number and type of gauge may vary from application to application. For example, the load cell may include only a single strain gauge or a pair of strain gauges. As another example, the load cell may be a different type of load cell, such as a piezoelectric load cell or a capacitive load cell. The strain gauges 28a-d of this embodiment are electrically arranged in a full Wheatstone bridge. Although the illustrated embodiment includes a full Wheatstone bridge, the electrical arrangement may vary from application to application. For example, the strain gauge(s) may alternatively be arranged in a quarter-bridge or half-bridge. As another example, the strain gauges may be arranged in a circuit configuration that does not include a Wheatstone bridge, such as other bridge or non-bridge arrangements. The load cell 16 shown in FIG. 2 is merely exemplary and the electronics module 22 may include any of a wide variety of alternative load cell types and load cell configurations.

The load cell carrier 16 of the illustrated embodiment is mounted transversely across the sled housing 14. In this embodiment, one end of the carrier 26 is fixed to the sled housing 14 and the other end is fixed to the linkage 18. As a result, the drag forces apply a force that is normal to the longitudinal extent of the carrier 26, which creates a bending moment on the carrier 26. In the illustrated embodiment, the carrier 26 is mounted to the sled housing 14 by a housing bracket 40 and to the linkage by a linkage bracket 42. The housing bracket 40 may be secured to the sled housing 14 and to the carrier 26 by fasteners (not shown), adhesive or other mechanisms. The linkage bracket 42 may be configured to extend from the free end of the carrier 26 to the approximate center of the sled housing 14. This allows the linkage 18 to be secured to the sled housing 14 at the approximate center of the sled housing 14, which helps to reduce canting of the sled housing 14 on the conveyor belt 204 during use.

In the illustrated embodiment, the electronics module 22 includes an amplifier 34 that amplifies the electrical signals generated by the load cell 16. The amplifier 34 is configured to provide the amount of gain desired to bring the load cell 16 output to a level appropriate for analysis by the controller 20. In this embodiment, the amplifier 34 is an instrumentation amplifier that includes a differential amplifier with input buffer amplifiers. Although the amplifier of this embodiment is an instrumentation amplifier, the amplifier may be essentially any type of amplifier capable of amplifying the output of the load cell to the desired level. In applications where a controller can analyze the load cell output without an amplifier, the amplifier can be eliminated. For example, the system may be implemented with a controller having internal amplifying capabilities or is specially configured to analyze small electrical signals.

In the illustrated embodiment, the electronics module 22 includes a controller 20 that receives input from the load cell 16 and produces an output signal intended to drive control the lubrication system 100. The controller 20 may be programmed to analyze the load cell 16 input to determine when the coefficient of friction exceeds a threshold value and, when it does, trigger the application of additional lubrication. In the illustrated embodiment, the controller 20 is configured with a digital output intended to control the lubrication system 100. The digital output may be coupled to a relay (not shown) or directly to a solenoid (not shown) that actuates the lubrication system. The relay or solenoid used to operate the lubrication system 100 may be a generally a conventional component. The output may be coupled to an amplifier (not shown), if desired. For example, if the controller 20 output signal is not of a level sufficient to directly operate a relay or solenoid, the electronics module 22 may include an output amplifier (not shown) capable of amplifying the output signal to the desired level. In the illustrated embodiment, the controller 20 is electronically connected to the relay or solenoid (not shown) by suitable wiring (not shown). Alternatively, the output signal may be transmitted wirelessly to a receiver located remote from the sled housing 14. For example, the electronics module 22 may include a wireless transmitter (or transceiver) (not shown) and the lubrication system 100 may include a wireless receiver (or transceiver) (not shown). In use, the controller 20 may communicate the output signal wireles sly and the receiver may receive the signal and operate the relay or solenoid appropriately. If wireless communications are provided, the transmitter and receiver may operate using essentially any suitable communications protocol, such as a Bluetooth protocol, a WiFi protocol, a Zigbee protocol or a Near-Field Communications protocol.

In the illustrated embodiment, the controller 20 is configured to operate the lubrication system 100 for a predetermined period of time. For example, the lubrication system 100 may be tested in advance to determine how long it should be run to reach an appropriate level of lubrication. As an alternative, the controller 20 may apply lubrication until the COF monitoring system 12 determines that the COF is within desired parameters. For example, the controller 20 may operate the lubrication system 100 for a relatively short period of time, wait for the lubrication to spread, measure the COF and repeat the process if the measured COF is not within the desired parameters.

Although the controller 20 of the illustrated embodiment directly controls the lubrication system 100, the output signal from the controller 20 may be sent to a separate lubrication controller (not shown) that uses it as a trigger signal that directs the lubrication controller to operate the lubrication system in accord with its own programming.

As noted above, the COF monitoring system 10 may be used in applications other than lubrication control application. In alternative applications, the controller 20 may be programed to implement the appropriate action(s), such as storing measurements in applications where a historical database is being produced or to trigger flags when the system is intended to determine fault conditions or to trigger repair or maintenance.

In the illustrated embodiment, the power source 24 is a battery, which is disposed within the sled housing 14. Alternatively, the power source 24 may be essentially any electrical energy storage devices, such as a super capacitor or other high capacity capacitor. As an alternative to an on-board power supply, the electronics module 22 may receive wired or wireless power from an outside power source. For example, the electronics module 22 may receive power from a transformer or other power supply that is coupled to wall power.

In the illustrated embodiment, the linkage 18 is a wire or string capable of holding the sled housing 14. For example, the linkage 18 may be a strand of monofilament fishing line or a braided fishing line. One end of the linkage 18 may be tied or otherwise fastened to a fixed structure adjacent the conveyor belt 204 and the other end may be tied or otherwise fastened to the linkage bracket 42. The linkage 18 may alternatively be essentially any structure capable of being affixed on one end to the load cell 16 and on the other end to a fixed structure adjacent the moving surface to be monitored.

In the illustrated embodiment, the lubrication system 100 generally includes lubricant supply lines 102, a pair of lubrication nozzles 104a-b, a lubricant pump (not shown) and a relay (not shown) that allows the controller 20 to activate the pump. The cones 106a-b shown in FIG. 1 represent the discharge of lubricant onto the belt in the form of a cone-shaped spray. In operation, the controller 20 is capable of closing the relay to activate the pump. Upon activation, the pump dispenses lubricant through the nozzles 104a-b onto the top surface of the conveyor belt 204. The illustrated lubrication system 100 is merely exemplary and the system 100 may be replaced by essentially any alternative lubrication system capable of selectively dispensing lubrication. For example, the number and location of the nozzles 104a-b may vary from application to application. As another example, the lubricant pump may be replaced by another source of motivation, such as compressed air.

General Operation.

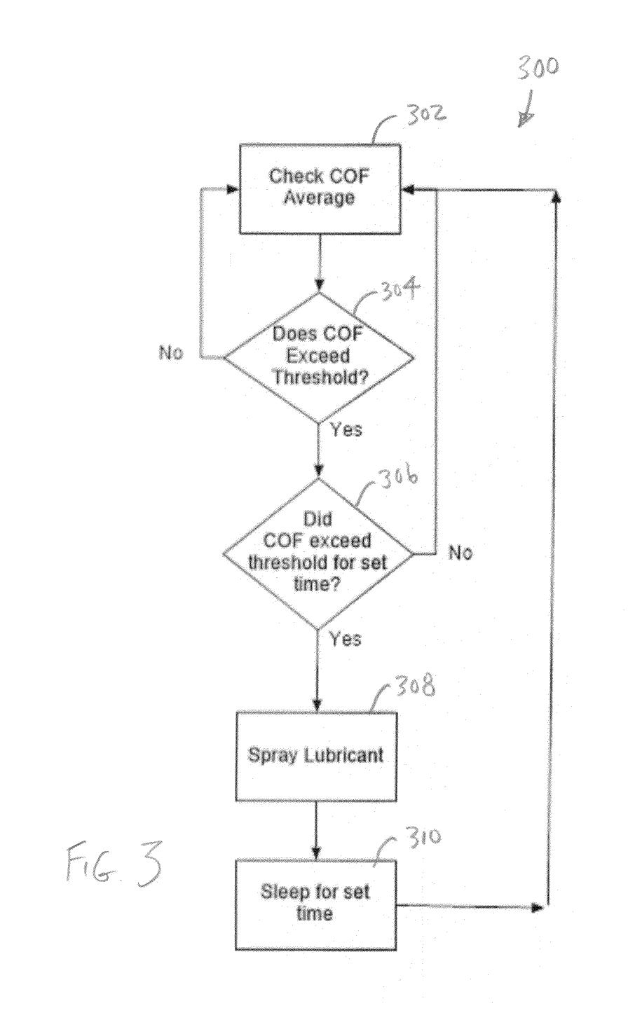

Operation of the embodiment of FIGS. 1-2 will now be described with reference to FIG. 3. In the illustrated embodiment, the system 10 is set up by fixing one end of the linkage 18 to a structural component adjacent to the conveyor belt 204. For example, the linkage 18 may be fastened to a conveyor system frame member F (See FIG. 1). The sled housing 14 is then placed on the conveyor belt 204. The controller 20 can then be activated.

Once activated, method 300 begins and the controller 20 enters “monitor mode.” In monitor mode, the controller 20 periodically measures the COF between the belt 204 and the sled housing 14 (block 302). More specifically, the controller 20 obtains and analyzes electrical signals from the load cell 16 to produce a COF average. In the illustrated embodiment, the controller 20 takes a series of 1000 sequential measurements and computes the COF average. The frequency and number of measurements included in the average may vary from application to application. If desired, a single COF measurement may be used in place of an average value determine from a plurality of COF measurements.

At block 304, the controller 20 determines whether the COF average exceeds the threshold value. If the computed COF average does not exceed a threshold value, control returns to block 302 and the controller 20 continues to obtain and analyze electrical signals from the load cell 16. The threshold value may be predetermined through testing, for example, at a manufacturing or testing facility or in the field.

If the COF average does exceed the threshold value, control passes to block 306 and the controller 20 continues to obtain and analyze electrical signals from the load cell 16 to determine if the COF average remains above the threshold for a desired period of time. This period of time may be predetermined through testing, for example at a manufacturing or testing facility or in the field. The sensitivity of the system 10 may be controlled by adjusting this period of time. If not, control passes to block 302 and the controller 20 returns to obtaining and analyzing electrical signals from the load cell 16.

If the COF average remains above the threshold for the requisite period of time, control passes to block 308 and the controller 20 initiates operation of the lubrication system 100. As noted above, the controller 20 may send an output signal that activates the lubrication system 100. For example, the controller 20 may send an output signal that operates a relay (not shown) that activates the lubrication system 100 or that controls a solenoid (not shown) that activates the lubrication system 100. As another example, the controller 20 may send an output signal to a controller in the lubrication system 100, and the lubrication system controller may operate the lubrication system 100. The controller 20 may send the output signal for a period of time coincident with the amount of time that it is desirable for the lubrication system 100 to operate. For example, if it is predetermined that it takes approximately 5 seconds of spray for the lubrication system 100 to adequately lubricate the conveyor belt 204, the controller may be configured to operate the lubrication system 100 for 5 seconds. This may be achieved in embodiments that include a relay or solenoid by driving the output signal high for a period of 5 seconds.

Once the lubrication system 100 has operated for the desired period of time, the monitor 12 enters a “delay mode” (See block 310). The delay mode is intended to provide a period of time during which the lubricant can spread over the belt 204. Taking COF measurements before the lubrication has had sufficient time to spread may lead to incorrect readings. The length of the delay period may be predetermined, for example, in a manufacturing or testing facility or during set up of the system 10 in the field. As an alternative, the monitor 12 may continue to take COF readings during the delay mode, but may ignore those readings until they have stabilized or until a period of time has lapsed.

In the embodiment described above, the controller 20 operates the lubrication system 100 for a period of time intended to fully lubricate the belt 204 once the COF average exceeds the threshold for a sufficient period of time. As an alternative to attempting to fully lubricate the belt in a signal activation of the lubrication system, the controller 20 may be configured to apply small amounts of lubrication until the desired COF has been reached. For example, the controller 20 may: (a) apply lubrication for 1 second, (b) delay for a period of time to allow the lubrication to spread, (c) determine the COF average after the delay period and (d) apply lubrication again if the COF average remains above the threshold. The process may be repeated until the COF average has reached the desired value.

As can be see, the present invention includes a variety of parameters that can be adjusted to tune operation of the device to different applications. For example, the Coefficient of Friction parameter that triggers the application of lubricant can be adjusted based on the moving surface being monitored, the Spray Length parameter can be selected to provide the desired amount of lubricant and the Delay time parameter can be adjusted to control the amount of time the system waits for lubrication to distribute before making further COF measurements. The system can be implemented with a tunable parameter that determines over how long of a period the average COF is measured. These and other system parameters may be adjusted from application to application to provide appropriate operation in a wide range of applications.

### Select Alternative Embodiments

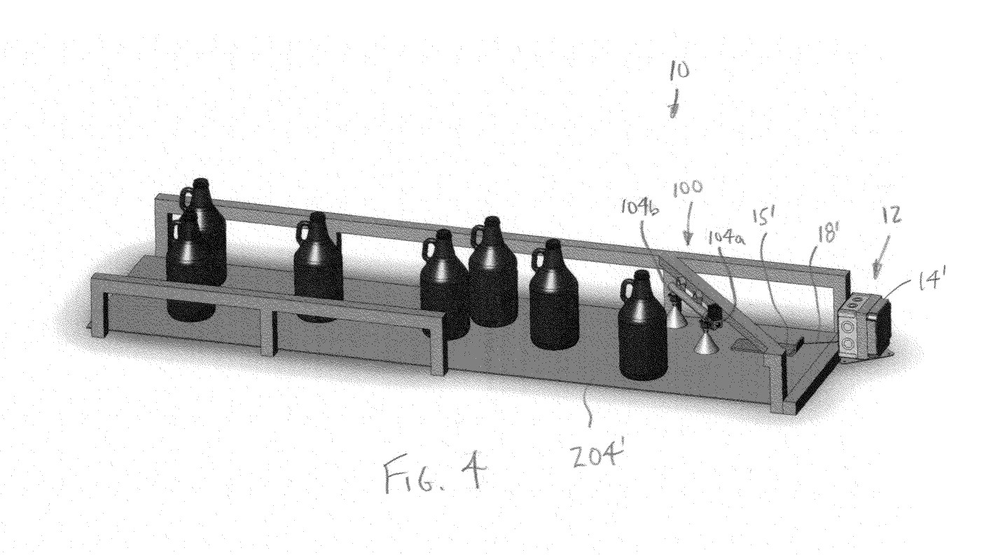

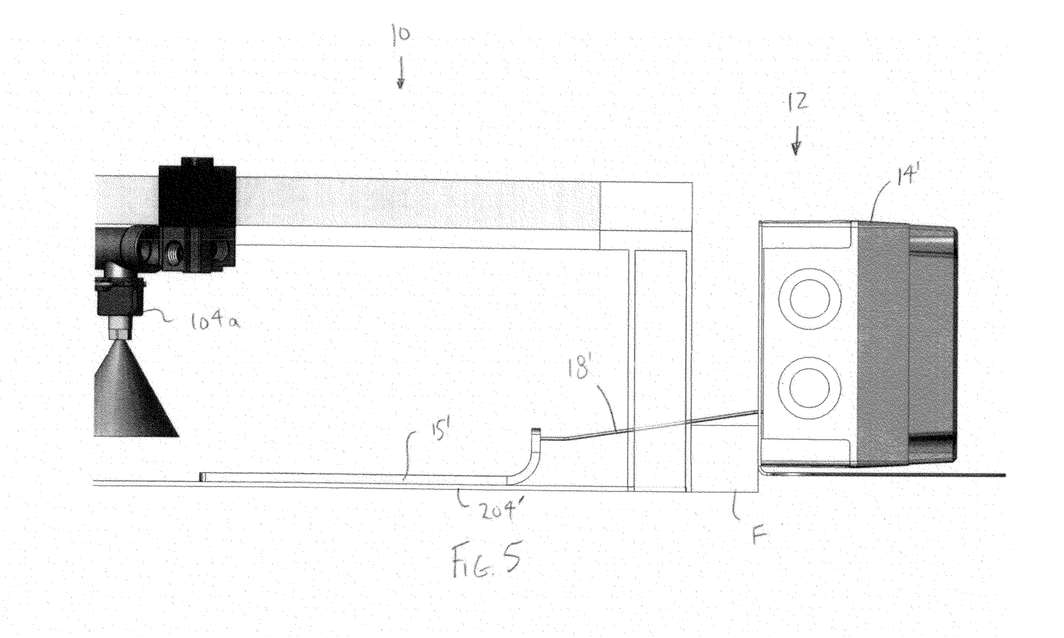

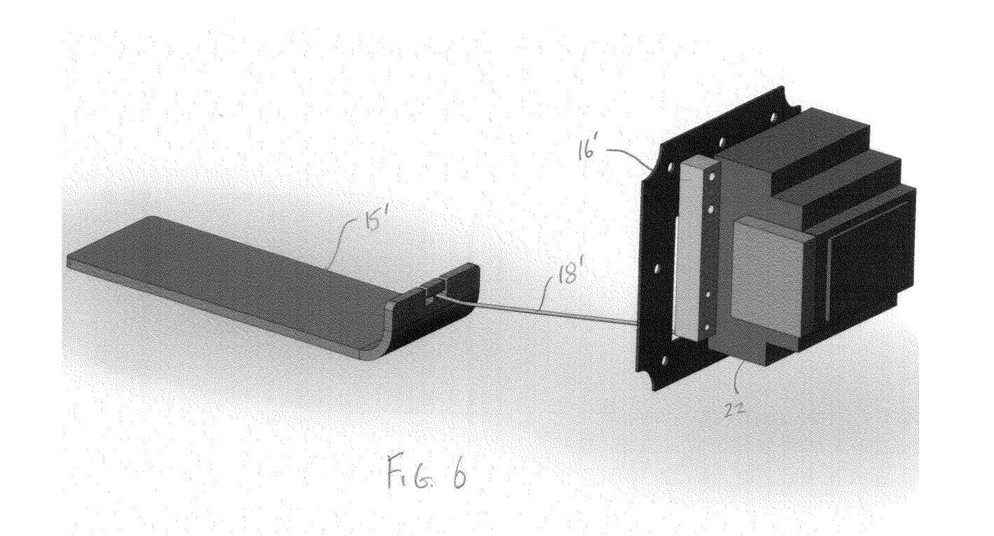

In the embodiment of FIGS. 4-6, the COF monitoring system 10 has a monitor 12 that includes a sled housing 14 that is placed on the moving surface S and houses the electronics module 22. In an alternative embodiment shown in FIG. 4, the system 10′ includes a monitor 12′ in which the electronics module 22′ is contained in a housing 14′ that is mounted to a fixed structure. For example, the housing 14′ may be mounted to a frame member of the conveyor system adjacent to an end of the conveyor belt 204′. In this embodiment, the monitor 12′ includes a weighted sled 15′ that is placed on the moving surface S and coupled to the electronics module 22′ by a linkage 18′ (See FIGS. 4 and 5). The linkage 18′ is coupled to the load cell 16′ so that the drag force of the sled 15′ with respect to the moving surface is communicated to the load cell 16′. For example, as shown in FIG. 6, this embodiment may incorporate a load cell 16′ that is essentially identical to the load cell 16 of system 10, with one end mounted to the housing 14′ and the opposite end mounted to the linkage 18′ so that force applied by the linkage 18′ creates a bending moment on the load cell 16′. As a result, the electronics module 22′ measures the drag force between the sled 15′ and the moving surface, which is representative of the COF between the weighted sled 15′ and the moving surface S. The design and configuration of the weighted sled 15′ may be varied form application to application as desired. For example, the weight of the sled 15′ and the shape of the bottom surface of the weighted sled may be varied to provide appropriate interaction with the corresponding conveyor belt 204′. In this embodiment, the electronic module 22′ may receive power via a wired connection rather than a battery or wireless power system. This embodiment may also simplify routing of the output signal to the lubrication system 100 (if included) as wires can be used for this purpose without the complexities associated with connecting wires to a sled housing 14 that is riding over the moving surface S. Except as otherwise noted, the system 10′ is essentially identical to system 10 described above and the controller 20′ may be operated in substantially the same ways as described above. To facilitate disclosure, the reference numerals used in FIGS. 4-6 generally correspond with those of FIGS. 1-2, except that they are denoted with a single prime symbol. Accordingly, unless otherwise stated, elements in FIGS. 4-6 correspond with elements in FIGS. 1-2 bearing the same base number.

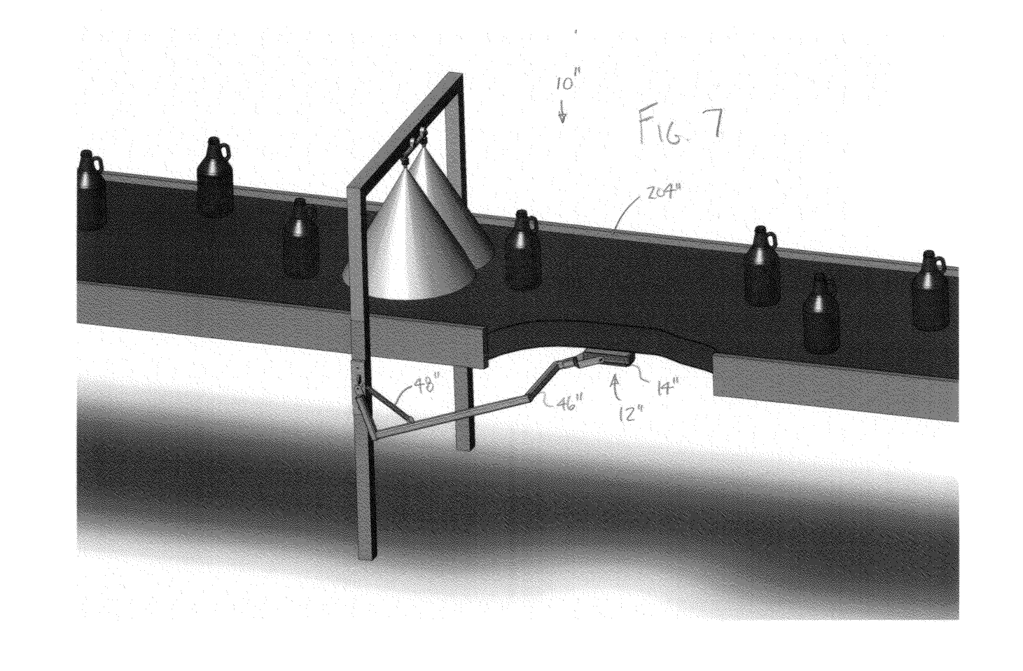

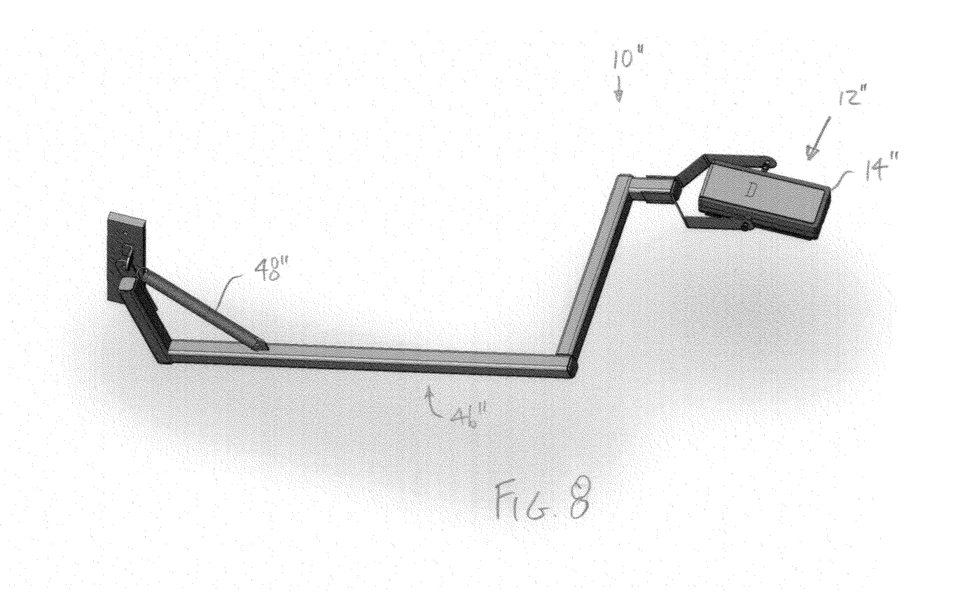

In the embodiments described above, the monitor 12 or sled 15′ is positioned on top of the moving surface S and the system relies on gravity to provide a force normal to the direction of movement of the surface. In alternative embodiments, the sled housing or sled may engage the moving surface from other directions. In the context of a conveyor belt, the monitor 12 may engage the conveyor belt at essentially any location at which the appropriate surface is exposed, including the top of the conveyor, the bottom of the conveyor, a take-up portion of the conveyor or and end portion of the conveyor. One example of this alternative is shown in the embodiment of FIGS. 7-13. In the embodiment of FIGS. 7-13, the COF monitoring system 10″ includes a monitor 12″ having a housing 14″ that engages the conveyor belt 204″ from the undersurface. This configuration may be particularly useful in applications where there limited space available on top of the moving surface. In this embodiment, the monitor 12″ is mounted to a lift arm 46″ that urges the monitor 12″ into contact with the undersurface of the conveyor belt 204″ (See FIG. 8). The lift arm 46″ of this embodiment includes a spring 48″ that is coupled between the frame 208″ of the conveyor system 200″ and the lift arm 46″ to provide an upward force to the lift arm 46″. The spring force may vary from application to application, as desired. Although the lift arm 46″ of this embodiment is mounted to the frame on a single side of the conveyor system 200″, the lift arm 46″ may be mounted to both sides in alternative applications. In alternative embodiments, the lift arm 46″ may be replaced by essentially any structure capable of providing appropriate upward force on the sled housing 14″. If desired, a backing plate (not shown) may be positioned above the conveyor belt 204 adjacent to the interface region of the sled housing 14″ to resist excessive upward movement of the conveyor belt 204 in response to the upward force on the sled housing 14″.

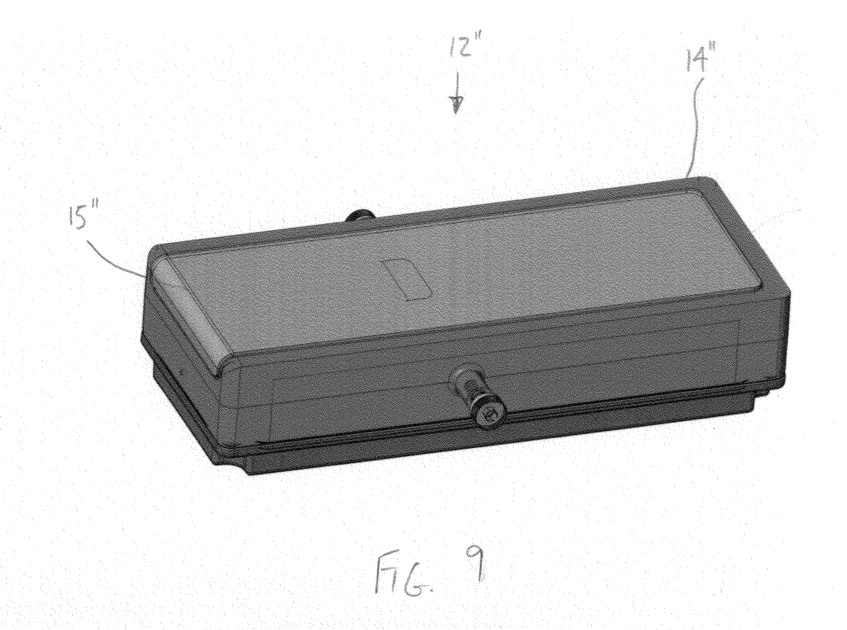

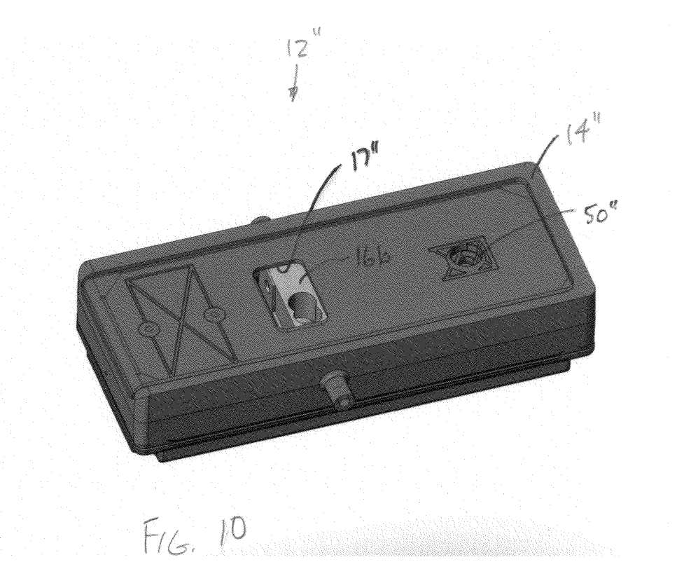

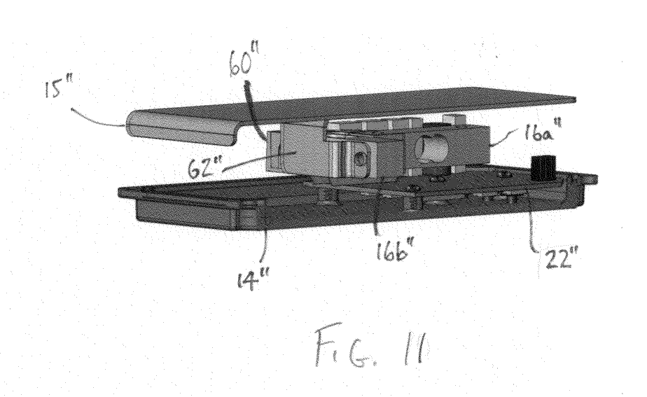

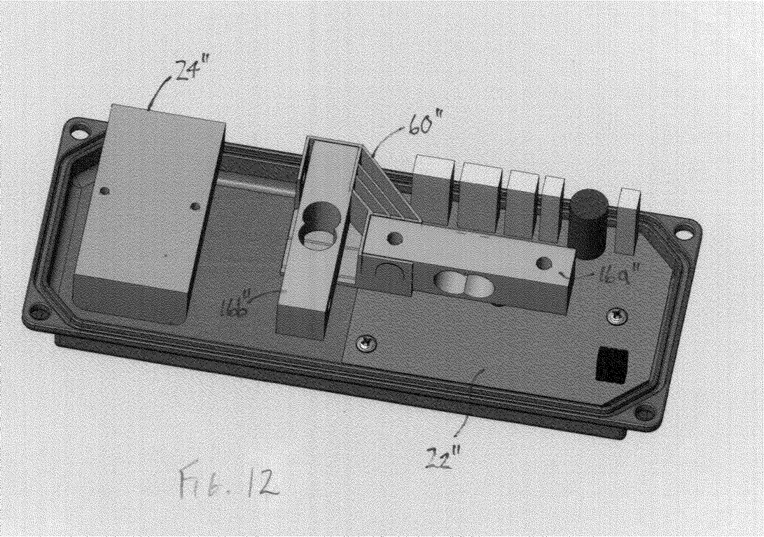

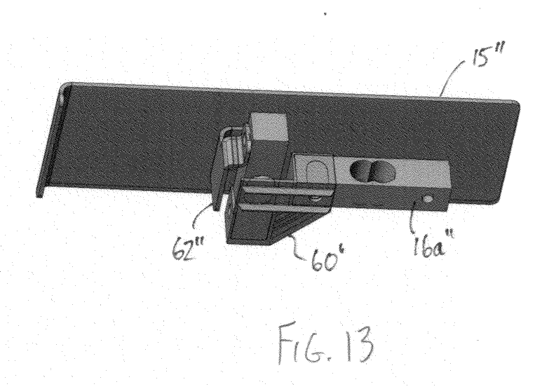

In this embodiment, the monitor 12″ includes a housing 14″ that houses the electronics module 22″ and a drag plate 15″ that engages the conveyor belt 204″ (See FIG. 9). The drag plate 15″ is essentially free floating with respect to the housing 14″ so that it can move relative to the housing 14″ in response to external forces, including normal and tangential forces as described in more detail below. FIG. 10 shows the monitor 12″ with the drag plate 15″ removed, thereby an aperture 17″ in the housing 14″. The linkage 18 is eliminated from this embodiment and the drag plate is operatively joined to the electronics module 22″ via a pair of load cells 16a″ and 16b″. FIG. 11 shows the monitor 12″ with portions of the housing 14″ removed, thereby showing the connection between the drag plate 15″ and the load cells 16a″ and 16b″. As perhaps best shown in FIGS. 12 and 13, load cell 16a″ is arranged to measure the normal force between the housing 14″ and the drag plate 15″ and load cell 16b″ is arranged to measure the tangential force between the housing 14″ and the drag plate 15″. As such, load cell 16a″ provides electrical signals representative of the amount of force being applied to the housing 14″ in the normal (or, in this case, upward) direction and load cell 16b″ provides electrical signals representative of the drag force between the drag plate 15″ and the conveyor belt 204″. In this embodiment, the normal force load cell 16a″ is fixed to the housing 14″ on one end and is cantilevered with the opposite end being free from the housing 14″. The housing 14″ of this embodiment may include a screw boss 50″ configured to receive a screw (not shown) for securing the load cell 16a″ to the housing 14″. The normal force load cell 16a″ extends in a direction generally parallel to the direction of movement of the conveyor belt 204″ and is arranged to measure bending forces applied in the vertical direction. The tangential force load cell 16b″ is coupled between the normal force load cell 16a″ and the drag plate 15″. More specifically, in this embodiment, one end of the tangential force load cell 16b″ is fixed to the free end of the normal force load cell 16a″ by a bracket 60″ and the opposite end of the tangential force load cell 16b″ is engaged with the drag plate 15″ by a plate bracket 62″. In this embodiment, the tangential load cell 16b″ extends in a direction generally perpendicular to the normal force load cell 16a″ and is arranged to measure bending forces created by drag between the drag plate 15″ and the moving conveyor belt 204″. As perhaps best shown in FIG. 13, the drag plate 15″ and the plate bracket 62″ are coupled so that forces applied to the drag plate 15″ are communicated to the load cells 16a″ and 16b″. In this embodiment, tangential forces are communicated from the drag plate 15″ to the plate bracket 62″ and the free end of the tangential load cell 16b″. Similarly, normal forces are communicated from the drag plate 15″ to the plate bracket 62″, the tangential load cell 16b″ and the load cell bracket 60″ to normal load cell 16a″. To facilitate disclosure, the reference numerals used in FIGS. 7-13 generally correspond with those of FIGS. 1-2, except that they are denoted with a double prime symbol. Accordingly, unless otherwise stated, elements in FIGS. 7-13 correspond with elements in FIGS. 1-2 bearing the same base number.

In this embodiment, the controller 22″ may be configured to compare the normal force measurements and the tangential force measurements to determine when action, such as lubrication, should occur. In some applications, the ratio between these two numbers may alone be sufficient to determine when to take action. In other applications, calculations based on the normal force and the tangential force may be used to determine when to take action. In yet other applications, the controller 22″ may include a look-up table (or other form of data storage) that provides action thresholds for different combinations of normal force and tangential force.

In this embodiment, the monitor 12″ engages the conveyor belt from below on the return portion of the belt's travel, it should be noted that the present invention may be configured to engage a conveyor belt or other moving surface from essentially any direction, including the top, bottom or ends. When it is desirable to engage an end, the monitor may be urged into the conveyor belt at a point where the belt passes over an end roller. This will allow the roller to function as a backing structure that prevents the conveyor belt from giving under the force of the monitor. The illustrated embodiments show the present invention in use on horizontal conveyors, but the present invention may also be used with inclined conveyors.

Although the present invention is described in connection with a COF monitoring systems 10, 10′ and 10″ that control lubricant application, the present invention is not limited to use in connection with the application of lubricant. Instead, the present invention may be used more broadly to implement essentially any system for dynamically controlling the frictional forces between at least one object and at least one moving surface based on measurement of the tangential force between the moving surface and an object in contact with the moving surface. Examples of alternative actions may include: measuring belt wear, measuring belt cleanliness. The system may be configured to make a logical decision to adjust essentially any adjustable parameter within the system that may have a material effect on the frictional forces between the object and the moving surface. This may include applying lubrication when the frictional forces exceed a threshold value.

In the illustrated embodiments, the systems generally includes a force measurement device, a controller (or microprocessor), and a switch controlled by the controller where the controller makes logical decisions to operate the switch based on the measured force and preprogrammed parameters accessible to the microprocessor. Although the switch (e.g. relay or solenoid) of the illustrated embodiments is used to operate the lubrication system, it may be used to operate essentially other system.

If desired, the system 10 may additionally or alternatively have the ability to record the state of parameters that may have a material effect of the frictional forces between the object and the moving surface. This information may be useful in developing methods for maintaining uniform lubrication or to otherwise assess the impact of various factors on the coefficient of friction.

In an alternative embodiment, the monitor 12 may be capable of transmitting the measured data to a remote device. This communication may occur over a wired or wireless communication link. For example, the controller 20 may be configured to communicate the data measured by the force measuring device to a remote device capable of making logical decisions to adjust parameters within the system with material effect on the frictional forces between the object and said moving surface. As another example, in data logging applications, the controller 20 may be configured to transmit measurements and system parameters to a remote device capable of storing the values of those parameters. The stored parameters may include the COF measurements and/or any other parameters that might be relevant to the frictional forces between the moving surface and an object placed on the moving surface.

As can be seen, the illustrated embodiments of the present invention provide systems for dynamically controlling the frictional forces between at least one object and at least one moving surface where the system (a) measures the tangential force between the object and the moving surface, (b) optionally measures the normal force between the object and the moving surface and (c) makes a logical decisions to adjust essentially any parameter(s) that might have a material effect on the frictional forces between the object and said moving surface.

In one embodiment, the system may include one load cell that measures the tangential force, and one load cell that measures the normal force. The system may compare the tangential force to the normal force and makes a logical decision to adjust essentially any parameter(s) within the system that might have a material effect on the relationship between tangential force and normal force of the object and the moving surface.

The present invention may be used to control the COF between a moving surface and objects that are to be carried on the moving surface. For example, the system may be used to control the COF between a conveyor belt and objects to be carried on the conveyor belt. To provide a more direct correlation between the COF measurements obtain by the system and the COF of the objects, the component of the COF monitoring system that is engaged with the moving surface may be manufactured from a material substantially similar to material from which the objects are manufactured. For example, with regard to some of the embodiments discussed above, the interface surface of the sled housing or the weighted sled may be manufactured from the same material as the objects. If desired, the interface surface may be interchangeable so that it can be changed from application to application depending on the nature of the objects being conveyed on the moving surface.

The present invention may use any of a wide variety of methods for interpreting the COF measurements and determining when action, such as lubrication, should be taken. For example, the system may include a controller that has access to a predetermined table containing correlation data between the frictional relationship between the component of the COF monitoring system that contacts the moving surface and the objects carried on the moving surface. The controller may use that table to make logical decisions to adjust essentially any parameter(s) that might have a material effect on the relationship between tangential force and normal force of the object and the moving surface within the system.

If desired, the COF monitoring system may include a user interface, such as a graphical display unit, to allow users to adjust parameters used by the system. For example, the user interface may allow a user to adjust any of the parameters associated with determine when action, such as lubrication, is to occur. This may include adjustment to the frequency of COF measurements, the threshold value against which COF measurements are compared, the number of COF measurements that are used to provide each COF average, the period of time the COF average must remain above the threshold value before action is taken and/or the length of the delay mode. As another example, the user interface may also allow a user to adjust parameters relating to the action to be taken. With regard to applications that include a lubrication system, this may include adjustments to the spray volume, the length of time the lubrication system is engaged or any other parameters that might impact lubrication.

The control algorithms described above are merely exemplary. It should be understood that the present invention may be implemented using a variety of alternative control algorithms. The control algorithms may vary depending on the system or apparatus being operated based on the COF measurements. For example, in the context of a system that controls operation of a lubrication system (e.g. lubrication system 100), rather than controlling operation based on a comparison of average COF measurements against a threshold value, the present invention may use alternative methods for obtaining data representative of COF and may analyze that data using alternative methods of analysis. Alternative methods for obtaining data may include essentially any method that allows acquisition of data representative of the COF between an engagement object (e.g. sled housing 14, sled 15′ and sled housing 14″) and the surface against which it is engaged (e.g. conveyor belt 204, 204′ and 204″). This may include collecting data representative of drag force between the engagement object and the surface. In some applications, the data representative of drag force may be combined with data representative of the normal force (i.e. the force urging the engagement object toward the surface) to obtain a COF estimate. In applications where the normal force is sufficiently constant, control decisions may be based on the drag force data and may not involve the normal force or the computation of an estimate of COF value. Alternative methods for analyzing the COF data may include essentially any method that allows an assessment of the current COF of the surface. In some applications, this may also include a determination of the action to be taken in response to the data representative of COF. For example, in some applications, the system may simply determine that action, such as the application of lubrication is required, is desirable based on data representative of COF. In such applications, the parameters of the action, such as the amount of lubricant to be applied, may be predetermined. However, in other applications, the system may also determine a parameter of the action to be taken, such as the volume of lubricant applied, based on the data representative of COF. For example, the system may determine the difference between the target and measured values of the data representative of the COF, and use that difference in determining the parameters of the action. In the context of a lubrication system, this may include increasing the amount of lubricant applied when the difference between the target and measured values is greater.

In one alternative embodiment, a load cell affixed to the engagement object and connected to a stationary object by a linkage emits an electric signal representative of the drag force. A controller receives the signal directly from the load cell, or through an amplifier, and periodically calculates the COF using the measured drag force and either the known normal force, or a normal force measured by another load cell that emits an electrical signal representative of the normal force. The controller determines an error value for each unit of time by calculating the difference between the COF at that time and a defined target COF. The controller stores the calculated errors, COFs, and associated time values in either volatile or non-volatile memory. Using the stored data, the controller calculates the adjustment required to any essential parameter(s) based on the value of the error at a given point in time, the average value of the error over a period of time, the time rate of change of the error, or any combination thereof. Two exemplary control algorithms which may be implemented are commonly referred to as PID (Proportional-Integral-Derivative) or PI (Proportional-Integral) controls algorithms, which we will be familiar to one skilled in the art of control systems. The controller may adjust parameters or transmit the required adjustments to another system in order to affect changes that substantially alter the COF of the moving surface.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

The embodiments of the invention in which an exclusive property or privilege is claimed are defined as follows:

1. A device for obtaining measurements representative of a coefficient of friction of a moving surface comprising:
an engagement object disposed in engagement with the moving surface;
a linkage securing said object to prevent said object from moving with the moving surface, whereby the moving surface moves with respect to said engagement object creating a drag force representative of the coefficient of friction of the moving surface; and
a force measuring component for obtaining a measurement representative of said drag force.

2. The device of claim 1 further including a housing, said force measuring component disposed within said housing.

3. The device of claim 2 wherein said force measuring component is a load cell, said load cell oriented substantially perpendicular to a direction of said drag force.

4. The device of claim 3 wherein said linkage is a flexible linkage coupling said engagement object to stationary structure.

5. The device of claim 4 wherein said housing is said engagement object.

6. The device of claim 5 further including a centering bracket coupling said linkage to said load cell.

7. The device of claim 1 further including a controller and a lubrication system, said controller configured to engage said lubrication system as a function of measurements from said force measuring component.

8. The device of claim 3 wherein said engagement object is a sled, said linkage joining said sled to said load cell, whereby said load cell obtains measurements representative of said drag force between said sled and the moving surface.

9. The device of claim 8 further including a controller and a lubrication system, said controller configured to engage said lubrication system as a function of measurements from said force measuring component.

10. The device of claim 1 wherein said linkage includes an arm biased toward the moving surface; and
wherein said engagement object includes a housing coupled to said arm and having an interface surface engaged with the moving surface, said arm applying a normal force to said housing urging said housing into the moving surface.

11. The device of claim 10 wherein said load measuring component is further defined as a first load cell; and
further including a second load cell for taking a measurement of said normal force.

12. The device of claim 11 wherein said interface surface is defined by a drag plate.

13. The device of claim 12 wherein said first load cell and said second load cell are operatively coupled together between said housing and said drag path.

14. The device of claim 13 further including a load cell bracket joining said first load cell and said second load cell.

15. The device of claim 14 wherein said first load cell is oriented substantially perpendicular to a direction of said drag force and said second load cell is oriented substantially perpendicular to said normal force.

16. A monitor and control system for a moving surface comprising:
a lubrication system for selectively applying a lubricant to the moving surface;
a monitor system including:
an engagement object disposed in engagement with the moving surface;
a linkage securing said object to prevent said object from moving with the moving surface, whereby the moving surface moves with respect to said engagement object creating a drag force representative of a coefficient of friction of the moving surface;
a force measuring component for obtaining a measurement representative of said drag force; and
a controller configured to selectively operate said lubrication system as a function of said measurement from said force measuring component.

17. The system of claim 16 wherein said lubrication system includes:
a supply of said lubricant;
a lubrication outlet configured to discharge said lubricant onto the moving surface; and
a switch for selectively engaging said lubrication system to apply said lubricant to the moving surface via said lubrication outlet.

18. The system of claim 16 wherein said force measuring component is a load cell, said load cell oriented substantially perpendicular to a direction of said drag force.

19. The system of claim 18 wherein said engagement object is a housing containing an electronic module including said controller and said load cell.

20. The system of claim 19 wherein said housing is disposed upon a substantially horizontal region of the moving surface where the housing is urged into engagement with said horizontal region of said moving surface by a force of gravity.

21. The system of claim 20 wherein said linkage is a flexible linkage coupling said housing to a stationary structure.

22. The system of claim 21 including a centering bracket coupling said linkage to said load cell.

23. The system of claim 16 wherein said engagement object is a sled; and
wherein said load cell is disposed in a housing, said linkage joining said sled to said load cell.

24. The system of claim 16 wherein said linkage includes an arm biased toward the moving surface; and
wherein said engagement object includes a housing coupled to said arm and having an interface surface engaged with the moving surface, said arm applying a normal force to said housing urging said housing into the moving surface.

25. The system of claim 24 wherein said load measuring component is further defined as a first load cell; and
further including a second load cell for taking a measurement of said normal force.

26. A method for maintaining lubrication on a moving surface, comprising the steps of:
obtaining a measurement representative of a coefficient of friction of the moving surface on a periodic basis;
determining when the measurement exceeds a threshold;
applying a lubricant to the moving surface when the measurement exceeds a threshold;
delaying for a predetermined period of time following said step of applying lubricant, whereby the lubricant is provided with time to become more evenly distributed before said obtaining step is repeated; and
repeating said steps of obtaining, determining, applying and delaying upon completion of said delaying step.

27. The method of claim 26 wherein said obtaining step is further defined as:
obtaining an average measurement by collecting a plurality of individual measurements and determining a numerical average of the measurements.

28. The method of claim 27 wherein said determining step is further defined as determining when the average of the measurement remains above a threshold for a predetermined period of time; and
said applying step is further defined as applying a lubricant to the moving surface when the average measurement exceeds a threshold for the predetermined period of time.

29. The method of claim 26 wherein said determining step is further defined as:
placing an engagement object in engagement with a moving surface;
securing the engagement object so that the engagement object does not move with the moving surface; and
obtaining measurements representative of a drag force between the engagement object and the moving surface.
